(19) United States
(12) Patent Application Publication
(10) Pub. No.: US 2024/0390448 A1
(43) Pub. Date: Nov. 28, 2024

(54) USE OF TRADITIONAL CHINESE MEDICINE COMPOSITION IN PREPARATION OF DRUGS FOR TREATING NEUROGENIC PULMONARY EDEMA

(71) Applicant: SHANDONG NEW TIME PHARMACEUTICAL CO., LTD., Linyi City, Shandong Province (CN)

(72) Inventors: Guimin Zhang, Linyi City, Shandong Province (CN); Jingchun Yao, Linyi City, Shandong Province (CN); Chenghong Sun, Linyi City, Shandong Province (CN); Honghua Li, Linyi City, Shandong Province (CN)

(21) Appl. No.: 18/695,998

(22) PCT Filed: Sep. 29, 2022
(86) PCT No.: PCT/CN2022/122723

§ 371 Date: Mar. 27, 2024

(30) Foreign Application Priority Data
Sep. 30, 2021 (CN) 202111161272.6

(51) Int. Cl. A61K 36/752; A61K 36/076; A61K 36/232; A61K 36/233; A61K 36/236; A61K 36/237; A61K 36/238; A61K 36/346; A61K 36/484; A61K 36/538; A61P 7/10; A61P 11/00

(52) U.S. Cl. CPC A61K 36/752; A61K 36/076; A61K 36/232; A61K 36/233; A61K 36/236; A61K 36/237; A61K 36/238; A61K 36/346; A61K 36/484; A61K 36/538; A61P 7/10; A61P 11/00

(57) ABSTRACT

Disclosed is a traditional Chinese medicinal composition for treating neurogenic pulmonary edema. The traditional Chinese medicinal composition is mainly prepared from Notopterygium root, angelicae pubescentis radix, poria cocos, radix saposhnikoviae, herba schizonepetae, rhizoma chuanxiong, platycodonis radix, radix bupleuri, common hogfennel root, fructus aurantii, and licorice root. The traditional Chinese medicinal composition can improve a lung index and lung wet weight/dry weight of a rat with neurogenic pulmonary edema, and has a therapeutic effect on the neurogenic pulmonary edema.

### TECHNICAL FIELD

The present invention belongs to the technical field of medicines, relates to new use of a traditional Chinese medicinal composition, and particularly relates to use of a traditional Chinese medicinal composition in the preparation of a drug for treating neurogenic pulmonary edema. The traditional Chinese medicinal composition comprises herba schizonepetae, radix saposhnikoviae, Notopterygium root, angelicae pubescentis radix, radix bupleuri, common hogfennel root, rhizoma chuanxiong, fructus aurantii, poria cocos, platycodonis radix, and licorice root.

### BACKGROUND ART

Pulmonary edema is caused by an increase in pulmonary extravascular fluid or an increase in internal capillary pressure due to fluid accumulation in the pulmonary interstitium or alveoli. Acute pulmonary edema is clinically mainly manifested by dyspnea, cyanosis, cough, colorless or pink foams while coughing, diffuse moist rales in the lungs, and severely respiratory failure and even death. The pulmonary edema has a high incidence rate and poor prognosis, and is a clinical common critical disease.

The pulmonary edema can be divided into cardiogenic pulmonary edema and non-cardiogenic pulmonary edema. The cardiogenic pulmonary edema is a sudden increase of the returned blood volume and the right cardiac output or a sudden and serious reduction of the left cardiac output, causing a great deal of blood to accumulate in the pulmonary circulation, such that the pulmonary capillary venous pressure is rapidly increased. When the pulmonary capillary venous pressure rises to exceed the colloid osmotic pressure in the pulmonary capillary, the hemodynamics in the capillary changes, extravasated blood is produced in the pulmonary circulation, the permeability of the pulmonary capillary wall is increased, and the liquid is filtered out through the pulmonary capillary wall to cause pulmonary edema.

The non-cardiogenic pulmonary edema is caused by a plurality of pathogenesis, but the pathogenesis is mainly the increased alveolar capillary blood pressure, increased alveolar capillary permeability, reduced plasma colloid osmotic pressure, or other factors such as return disorder of pulmonary lymph, increased negative pressure of tissue septum and the like. Neurogenic pulmonary edema refers to a clinical syndrome of acute pulmonary edema caused by acute pulmonary interstitial and alveolar fluid exudation due to craniocerebral injury or central nervous system diseases in the absence of primary diseases of the heart and lungs.

Clinically, according to different causes of pulmonary edema, antibiotics, adrenocortical hormone drugs, diuretics, vasodilators and the like are used for treating the pulmonary edema. But the drugs have different degrees of side effects, such as hypotension caused by the vasodilators, electrolyte disturbance caused by the diuretics and the like.

Although the name of pulmonary edema is not recorded in ancient medical literatures, similar symptoms of the pulmonary edema are the same as those of “thoracic fluid retention”, “dyspnea syndrome”, “lung distension”, “edema”, and “abnormal rising of qi and cough”. According to the main clinical manifestations of pulmonary edema and combining ancient and modern clinical understanding, the etiology of the pulmonary edema can be classified into three aspects of exogenous evil attacking the lungs and heart, and physical weakness due to chronic diseases.

A herba schizonepetae and radix saposhnikoviae granule is prepared by mutually matching herba schizonepetae, radix saposhnikoviae, Notopterygium root, angelicae pubescentis radix, radix bupleuri, common hogfennel root, rhizoma chuanxiong, fructus aurantii, poria cocos, platycodonis radix, and licorice root, has the effects of inducing sweat and relieving exterior syndrome, dispelling wind and eliminating phlegm, and clearing the lungs and relieving cough, has very good effects on a series of symptoms after used for treating exogenous wind-cold, is safe, and has few side effects.

### SUMMARY

The present invention provides a traditional Chinese medicinal composition in the preparation of a drug for treating neurogenic pulmonary edema. The traditional Chinese medicinal composition is prepared from herba schizonepetae, radix saposhnikoviae, Notopterygium root, angelicae pubescentis radix, radix bupleuri, common hogfennel root, rhizoma chuanxiong, fructus aurantii, poria cocos, platycodonis radix, and licorice root. The present invention further develops use of the traditional Chinese medicinal composition on the basis of the existing herba schizonepetae and radix saposhnikoviae granule product. On the basis of feedbacks of clinical use, the inventor carries out research and development of new use of the herba schizonepetae and radix saposhnikoviae granule and a formulation thereof. The experimental researches show that the herba schizonepetae and radix saposhnikoviae granule can obviously reduce a lung index and lung wet weight/dry weight of a rat with neurogenic pulmonary edema induced by oleic acid, relieve a pathological state of a lung tissue of the rat, and reduce the degree of lung tissue edema. The herba schizonepetae and radix saposhnikoviae granule can further obviously reduce lung wet weight/dry weight of a mouse with neurogenic pulmonary edema induced by spinal cord injury, relieve a pathological state of a lung tissue of the mouse, reduce the degree of lung tissue edema, and increase selection of a drug for clinically treating neurogenic pulmonary edema.

Herba schizonepetae is slightly warm in nature, has the effects of relieving exterior syndrome and dispelling wind due to warm-pungent nature, and can be widely used for a patient with wind-cold, wind-heat, and unobvious cold and heat. The herba schizonepetae can further promote eruption and eliminate sores, has a certain therapeutic effect on diseases such as sores, rubella, measles and the like, and further has an effect of relieving itching. In addition, the herba schizonepetae carbonized by stir-frying turns from warm-pungent to bitter and mild, has a certain bleeding-stopping effect, and can relieve symptoms such as hematochezia, postpartum hemorrhage, metrorrhagia and metrostaxis and the like.

Radix saposhnikoviae is pungent and warm in nature, has the effects of dispelling wind and relieving exterior syndrome, and has a certain conditioning effect on symptoms such as headache, anhidrosis, fever and the like caused by common cold due to wind-cold. The radix saposhnikoviae can further remove dampness and relieve pains, and can relieve diseases such as rheumatic arthralgia, bone joint ache and the like. In addition, the radix saposhnikoviae has the effect of relieving spasm and can relieve the symptoms of limb spasm. Modern pharmacological researches show that the radix saposhnikoviae can calm the mind, relieve pains and resist convulsion, and is commonly used for adjuvant therapy of tetanus.

Notopterygium root is considered to be pungent, bitter, and warm in nature, belongs to bladder and kidney channels, and can relieve exterior syndrome and dispel cold, clear wind-damp, and relieve pains. Firstly, the Notopterygium root is pungent, can dispel cold, evil and stasis and promote qi and blood circulation, and can treat wind-cold exterior syndrome of cold due to warm nature. Secondly, the Notopterygium root is warm and dry, and can dispel dampness, relieve exterior syndrome, clear wind-damp, and treat anemofrigid-damp arthralgia of the upper part of the body. The Notopterygium root further has an analgesic effect, and can be used for external application and oral administration. Modern pharmacological researches show that volatile oil of the Notopterygium root has obvious antipyretic and analgesic effects and has an inhibitory effect on dermatophytes.

Angelicae pubescentis radix can regulate meridians and collaterals, promote qi and blood circulation, dispel wind and remove dampness, and relieve symptoms such as joint ache, joint flexion and extension difficulty, hand and foot numbness and the like caused by anemofrigid-damp arthralgia. Besides, the angelicae pubescentis radix can further be used for relieving symptoms such as wind-fire toothache, wind-induced headache and the like. The angelicae pubescentis radix is clinically commonly used for treating rheumatic arthritis, rheumatoid arthritis, sciatica, cervical spondylosis, scapulohumeral periarthritis, etc.

Radix bupleuri belongs to liver channel, has the efficacy of soothing liver and relieving depression, and has certain conditioning effects on symptoms such as distending pain in chest and hypochondrium, irregular menstruation, depression, distending pains in breast and the like. The radix bupleuri is slightly cold in nature, can further bring down a fever, and can relieve fever caused by diseases such as acute bronchitis, acute tracheitis, viral pneumonia and the like. In addition, the radix bupleuri has an effect of tonifying yang and can relieve qi deficiency and organ prolapse. The pharmacological researches show that the radix bupleuri has the effects of calming the mind and calming pains, and can further protect the liver and relieve liver injury.

Common hogfennel root is bitter and pungent, and slightly cold in nature, belongs to enters lung channel, has effects of depressing qi and reducing phlegm, can treat symptoms such as yellow phlegm, excessive and sticky phlegm, fever and thirst, shortness of breath and the like caused by phlegm-heat stagnation in the lungs and loss of ascent and descent of lung qi, can help clear fire in the lungs, relieve the adverse rise of lung qi and lower the fire, and can further assist in treating respiratory tract infectious diseases such as bronchitis, pneumonia and the like. The common hogfennel root further has effects of dispelling wind and clearing heat, is suitable for a patient suffering from wind-heat, and can help dispel wind and heat, and relieve symptoms such as headache, dysphoria, insomnia and the like.

Rhizoma chuanxiong is a medicinal material commonly used in traditional Chinese medicine clinically, mainly grows in the mild climatic environment, is warm in nature, and belongs to liver, gallbladder and pericardium channels. The rhizoma chuanxiong has effects of promoting blood circulation, removing blood stasis, alleviating pain expelling wind, and regulating menstruation for patients with pains in chest and hypochondrium, rheumatic arthralgia, menoxenia, and traumatic injury. In addition, the volatile oil component contained in the rhizoma chuanxiong has a certain inhibitory effect on cerebral activity, avoids overexcitation of human body, and calms the mind.

Fructus aurantii is considered to be bitter by traditional Chinese medicine, belongs to spleen and stomach channels, and can regulate qi and dispel middle jiao qi, and activate stagnancy and relieve distension. The fructus aurantii is a dried immature fruit of orange and has the nature, taste and channel similar to those of immature bitter orange. But the effect is relatively mild. Since the fructus aurantii can be used for regulating qi and dispelling middle jiao qi, and activating stagnancy and relieving distension, it can be used for treating qi stagnation in chest and hypochondrium, swelling pains in the hypochondrium, food dyspepsia, and flatulence and prolapse. The fructus aurantii is widely used in food therapy and mainly used for strengthening spleen to promote digestion, and can relieve the symptoms of fullness and discomfort. The modern pharmacological researches show that the fructus aurantii can regulate gastrointestinal movement and excite intestinal smooth muscles.

Poria cocos has a mild nature, belongs to a spleen channel, can tonify spleen after being soaked in water for drinking, and has a certain conditioning effect on diseases such as spleen deficiency, poor appetite, loose stool, diarrhea and the like. Secondly, the poria cocos belongs to a heart channel, further has an effect of calming the heart, can relieve palpitation, insomnia, malaise and other diseases, and is beneficial to improving sleep quality. In addition, the poria cocos soaked in water for drinking has the effects of clearing damp and promoting diuresis, and can relieve symptoms such as edema, oliguria, difficult urination and the like.

Platycodonis radix is bitter and pungent, and mild in nature, and belongs to a lung channel. The platycodonis radix is commonly used for treating cough with excessive phlegm, chest distress, pharyngalgia, hoarseness, pulmonary abscess, and purulent sputum. Platycodonis radix in water has both effects of reducing blood lipid and blood sugar.

Licorice root is sweet and mild and belongs to heart, lung, spleen and stomach channels. The licorice root has effects of tonifying qi and restoring pulse, and the enhanced effect of tonifying qi after stir-fried with honey. The licorice root can be used for adjuvantly treating heart-qi deficiency, palpitation, and pulse depression. The licorice root is sweet and cool, can clear heat and remove toxicity, and can be used for adjuvantly treating sore throat, carbuncle, and sore-toxin. The licorice root is mild, can resolve phlegm and stop cough, and replenish qi and moisten lung, and can be used for treating cough due to wind-cold evil, wind-heat evil, cold phlegm evil, damp phlegm evil, and lung dryness. The licorice root acts on middle jiao, has effects of strengthening the spleen and stomach, and relieving pains, and can also be used for treating qi deficiency of the spleen and stomach, debilitation, poor appetite, loose stool, abdominal distention, or acute limb pains.

The present invention provides the specific composition of the traditional Chinese medicinal composition which is mainly prepared from the following raw materials in parts by weight:
5-30 parts of herba schizonepetae and 5-30 parts of radix saposhnikoviae.

The traditional Chinese medicinal composition is mainly prepared from the following raw materials in parts by weight:
10-20 parts of herba schizonepetae and 15-25 parts of radix saposhnikoviae.

The traditional Chinese medicinal composition is mainly prepared from the following raw materials in parts by weight:
5-30 parts of herba schizonepetae, 5-30 parts of radix saposhnikoviae, 5-30 parts of Notopterygium root,
5-30 parts of angelicae pubescentis radix, 3-25 parts of radix bupleuri, and 3-25 parts of common hogfennel root.

The traditional Chinese medicinal composition is mainly prepared from the following raw materials in parts by weight:
5-30 parts of herba schizonepetae, 5-30 parts of radix saposhnikoviae, 5-30 parts of Notopterygium root,
5-30 parts of angelicae pubescentis radix, 3-25 parts of radix bupleuri, 3-25 parts of common hogfennel root,
5-30 parts of rhizoma chuanxiong, 3-25 parts of fructus aurantii, 5-30 parts of poria cocos, and
3-25 parts of platycodonis radix.

The traditional Chinese medicinal composition is mainly prepared from the following raw materials in parts by weight:
5-30 parts of herba schizonepetae, 5-30 parts of radix saposhnikoviae, 5-30 parts of Notopterygium root,
5-30 parts of angelicae pubescentis radix, 3-25 parts of radix bupleuri, 3-25 parts of common hogfennel root,
5-30 parts of rhizoma chuanxiong, 3-25 parts of fructus aurantii, 5-30 parts of poria cocos,
3-25 parts of platycodonis radix, and 1-10 parts of licorice root.

The traditional Chinese medicinal composition is mainly prepared from the following raw materials in parts by weight:
10-20 parts of herba schizonepetae, 10-20 parts of radix saposhnikoviae, 10-20 parts of Notopterygium root,
10-20 parts of angelicae pubescentis radix, 5-20 parts of radix bupleuri, 5-20 parts of common hogfennel root,
10-20 parts of rhizoma chuanxiong, 5-20 parts of fructus aurantii, 10-20 parts of poria cocos,
5-20 parts of platycodonis radix, and 3-10 parts of licorice root.

The traditional Chinese medicinal composition is mainly prepared from the following raw materials in parts by weight:
12-18 parts of herba schizonepetae, 12-18 parts of radix saposhnikoviae, 12-18 parts of Notopterygium root,
12-18 parts of angelicae pubescentis radix, 10-18 parts of radix bupleuri, 10-20 parts of common hogfennel root,
10-20 parts of rhizoma chuanxiong, 10-20 parts of fructus aurantii, 10-20 parts of poria cocos,
10-20 parts of platycodonis radix, and 3-8 parts of licorice root.

The traditional Chinese medicinal composition is mainly prepared from the following raw materials in parts by weight:
15 parts of herba schizonepetae, 15 parts of radix saposhnikoviae, 15 parts of Notopterygium root,
15 parts of angelicae pubescentis radix, 15 parts of radix bupleuri, 15 parts of common hogfennel root,
15 parts of rhizoma chuanxiong, 15 parts of fructus aurantii, 15 parts of poria cocos,
15 parts of platycodonis radix, and 5 parts of licorice root.

The traditional Chinese medicinal composition is mainly prepared from the following raw materials in parts by weight:
15 parts of herba schizonepetae, 15 parts of radix saposhnikoviae, 15 parts of Notopterygium root,
15 parts of angelicae pubescentis radix, 15 parts of radix bupleuri, 10 parts of common hogfennel root,
10 parts of rhizoma chuanxiong, 10 parts of fructus aurantii, 10 parts of poria cocos,
10 parts of platycodonis radix, and 5 parts of licorice root.

The present invention provides a method for preparing the traditional Chinese medicinal composition. The preparation method mainly comprises the following steps: herba schizonepetae, radix saposhnikoviae, Notopterygium root, angelicae pubescentis radix, radix bupleuri, common hogfennel root, rhizoma chuanxiong, and fructus aurantii are firstly respectively distilled to extract a volatile oil for a standby application, the distilled medicinal residues and distilled rhizoma chuanxiong and fructus aurantii water solutions are collected for a standby application, and then other operations are performed.

Specifically, the preparation method mainly comprises the following steps:
step A: respectively distilling herba schizonepetae, radix saposhnikoviae, Notopterygium root, angelicae pubescentis radix, common hogfennel root, rhizoma chuanxiong, and fructus aurantii to extract a volatile oil for a standby application, and collecting distilled medicinal residues, and distilled rhizoma chuanxiong and fructus aurantii water solutions for a standby application;
step B: preparing the distilled rhizoma chuanxiong and fructus aurantii water solutions obtained in step A into a 10%-40% ethanol solution for a standby application;
step C: mixing poria cocos, and the distilled rhizoma chuanxiong and fructus aurantii medicinal residues obtained in step A, percolating the mixture with the ethanol solution obtained in step B, and collecting a percolate for a standby application;
step D: decocting radix bupleuri, platycodonis radix, licorice root, the distilled herba schizonepetae, radix saposhnikoviae, Notopterygium root, angelicae pubescentis radix, and common hogfennel root medicinal residues obtained in step A in water, and concentrating a decoction for a standby application; and
step E: mixing the percolate obtained in step C and the decoction obtained in step D, concentrating the mixture, and adding the volatile oils obtained in step A to obtain the traditional Chinese medicinal composition.

Further, the preparation method mainly comprises the following steps:
step A: respectively distilling herba schizonepetae, radix saposhnikoviae, Notopterygium root, angelicae pubescentis radix, common hogfennel root, rhizoma chuanxiong, and fructus aurantii to extract a volatile oil for a standby application, and collecting distilled medicinal residues, and distilled rhizoma chuanxiong and fructus aurantii water solutions for a standby application;
step B: preparing the distilled rhizoma chuanxiong and fructus aurantii water solutions obtained in step A into a 15%-30% ethanol solution for a standby application;
step C: mixing poria cocos, and the distilled rhizoma chuanxiong and fructus aurantii medicinal residues obtained in step A, percolating the mixture with the ethanol solution obtained in step B, and collecting a percolate for a standby application;
step D: decocting radix bupleuri, platycodonis radix, licorice root, the distilled herba schizonepetae, radix saposhnikoviae, Notopterygium root, angelicae pubescentis radix, and common hogfennel root medicinal residues obtained in step A in water, and concentrating a decoction into a thick paste for a standby application; and
step E: mixing the percolate obtained in step C and the thick paste obtained in step D, concentrating the mixture into a clear paste, and adding the volatile oils obtained in step A to obtain the traditional Chinese medicinal composition.

A fourth purpose of the present invention is to provide a traditional Chinese medicinal formulation containing the traditional Chinese medicinal composition. The traditional Chinese medicinal formulation is a clinically acceptable oral formulation.

The pharmaceutical composition of the present invention may be prepared from a conventional pharmaceutic adjuvant by using a conventional formulation technique.

The conventional pharmaceutic adjuvant is selected from one or more of an excipient, a lubricant, a binder, an emulsifier, a disintegrant, a stabilizer, and a corrigent.

The excipient is selected from one or more of lactose, sucrose, glucose, mannitol, sorbitol, starch, dextrin, crystalline cellulose, acacia gum, and glucan.

The lubricant is selected from one or more of magnesium stearate, calcium stearate, talc powder, superfine silica powder, boric acid, and sodium dodecyl sulfate.

The binder is selected from one or more of hydroxypropyl cellulose, hydroxypropyl methyl cellulose, polyvinylpyrrolidone, and polyethylene glycol.

The disintegrant is selected from one or more of low-substituted hydroxypropyl cellulose, carboxymethyl cellulose, sodium carboxymethyl starch, and cross-linked polyvinylpyrrolidone.

The emulsifier is selected from one or more of bentonite, magnesium hydroxide, aluminum hydroxide and sodium dodecyl sulfate.

The stabilizer is selected from one or more of methyl 4-hydroxybenzoate, benzyl alcohol, phenethyl alcohol, phenol, sorbic acid, and dehydroacetic acid.

The corrigent is selected from one or more of sucrose, spice, aspartame, and cyclodextrin.

Further, the clinically acceptable oral formulation is one or more of a pill, a capsule, a tablet, a granule, a mixture, or an oral liquid.

Furthermore, the oral formulation is a granule or a mixture.

The present invention further provides use of the traditional Chinese medicinal composition in the preparation of a drug for treating neurogenic pulmonary edema.

Neurogenic pulmonary edema (NPE) is a clinical syndrome characterized by acute pulmonary edema after central nervous system injury. The neurogenic pulmonary edema is mainly seen in central nervous system lesions such as spinal cord injury, intracranial hemorrhage, status epilepticus, meningitis, subarachnoid hemorrhage, bulbar lesion and the like, and usually occurs within 24-72 h after the onset of the central nervous system lesions.

The neurogenic pulmonary edema is different from acute respiratory distress syndrome. A patient with the acute respiratory distress syndrome is free of cardiogenic or pulmonary dysfunction, and rise of pulmonary extravascular hydrostatic pressure and oxygenation disorder caused by the central nervous system injury. One common point of the neurogenic pulmonary edema is acute severe central nervous system injury. The acute severe central nervous system injury is acute and rapid, sharply increases intracranial pressure, and causes a brain tissue to be compressed, ischemic, or damaged, thereby causing extreme excitement of the sympathetic nervous system, releasing catecholamine, and causing an abnormal increase of pulmonary extravascular hydrostatic pressure to lead to pulmonary edema.

Furthermore, the neurogenic pulmonary edema is pulmonary edema caused by craniocerebral injury or a central nervous system disease.

The neurogenic pulmonary edema is a pulmonary extravascular hydrostatic pressure rise and oxygenation disturbance caused by injury of central nervous systems such as medulla oblongata, hypothalamus, etc., such that a series of lung lesion syndromes appear. The neurogenic pulmonary edema is divided into two clinical subtypes, namely acute type and chronic type.

The clinical manifestations of the neurogenic pulmonary edema are atypical and easily confused with other pulmonary diseases. Acute dyspnea and progressive hypoxemia are the most important clinical manifestations of the neurogenic pulmonary edema. In the early stage of the neurogenic pulmonary edema, only some nonspecific manifestations, such as tachypnea, blood pressure rise, and pulse increase, may occur. In the late stage of the neurogenic pulmonary edema, typical manifestations, such as tachypnea, tachycardia, cyanosis, pink frothy sputum, pulmonary vesicular rale and wet rale and the like, similar to those of respiratory distress syndrome, and the general oxygen inhalation therapy is ineffective.

Further, the neurogenic pulmonary edema is selected from one or more of pulmonary edema caused by craniocerebral trauma, a surgery, subarachnoid hemorrhage, cerebral embolism, cerebral hemorrhage, brainstem infarction, an intracranial tumor, acute cerebral edema, epilepsy, and virus infection combined with a central nervous system lesion.

The neurogenic pulmonary edema is pulmonary edema caused by cerebrovascular accident, brain tumor, brain trauma, epileptic seizure, and other brain pathological changes, and is also called brain-derived pulmonary edema. The neurogenic pulmonary edema is a clinical syndrome characterized by acute pulmonary edema after central nervous system injury.

The craniocerebral trauma is a common injury occurring at the head, mostly due to falling and crushing, and then striking. The craniocerebral trauma often occurs in disasters, wars, or traffic accidents, and plays an important place in the trauma of each part of the whole body. The craniocerebral trauma can be classified into soft tissue (scalp) injury, head injury (fracture), and intracranial tissue (cerebral vessels and meninges) injury.

The surgery is a neurosurgery.

The subarachnoid hemorrhage refers to a clinical syndrome caused by rupture of a diseased blood vessel at the bottom of the brain or on the surface of the brain and direct inflow of blood into the subarachnoid space, and is also called primary subarachnoid hemorrhage.

The cerebral embolism refers to the blockage of blood vessels caused by various emboli (such as mural thrombus in the heart, atherosclerotic plaque, fat, tumor cells, fibrocartilage or air, etc.) in blood entering cerebral arteries along with blood flow. When collateral circulation cannot be compensated, ischemic necrosis of cerebral tissues in an artery blood supply area is caused and focal neurologic impairment occurs.

The cerebral hemorrhage refers to hemorrhage caused by rupture of blood vessels in non-traumatic brain parenchyma, and is mainly related to pathological changes of cerebral vessels, namely, closely related to hyperlipidemia, diabetes, hypertension, aging of blood vessels, smoking and the like. A patient with cerebral hemorrhage often suffers from sudden onset of diseases due to excited emotion and strenuous exertion and has a very high early mortality rate. Most survivors have sequelae such as dyskinesia, cognitive disorder, speech and swallowing disorder and the like in different degrees.

The brainstem infarction is one of cerebral infarction and is the most serious cerebral infarction. The brainstem infarction refers to ischemia of midbrain, pons and medulla oblongata caused by the stenosis or occlusion of the vertebrobasilar artery and branch vessels thereof due to arteriosclerosis, embolism, spasm, and inflammation, thereby causing corresponding nervous system symptoms and signs.

The intracranial tumor refers to a brain tumor, including a primary brain tumor of brain parenchyma and a secondary brain tumor metastasized intracranially from other parts of the body.

The epilepsy is a chronic disease of transient cerebral dysfunction caused by a sudden abnormal discharge of cerebral neurons.

Further, the neurogenic pulmonary edema is selected from one or more of pulmonary edema caused by craniocerebral trauma, a surgery, subarachnoid hemorrhage, cerebral embolism, cerebral hemorrhage, brainstem infarction, an intracranial tumor, acute cerebral edema, and virus infection combined with a central nervous system lesion.

Further, the neurogenic pulmonary edema is selected from one or more of pulmonary edema caused by craniocerebral trauma, a surgery, subarachnoid hemorrhage, cerebral embolism, cerebral hemorrhage, brainstem infarction, an intracranial tumor, acute cerebral edema, and epilepsy.

Further, the neurogenic pulmonary edema is selected from one or more of pulmonary edema caused by craniocerebral trauma, a surgery, subarachnoid hemorrhage, cerebral embolism, cerebral hemorrhage, brainstem infarction, an intracranial tumor, and acute cerebral edema.

Further, the neurogenic pulmonary edema is selected from one or more of pulmonary edema caused by craniocerebral trauma, a surgery, subarachnoid hemorrhage, cerebral embolism, cerebral hemorrhage, brainstem infarction, and an intracranial tumor.

Further, the neurogenic pulmonary edema is selected from one or more of pulmonary edema caused by craniocerebral trauma, a surgery, subarachnoid hemorrhage, cerebral embolism, cerebral hemorrhage, and brainstem infarction.

Further, the neurogenic pulmonary edema is selected from one or more of pulmonary edema caused by craniocerebral trauma, a surgery, subarachnoid hemorrhage, cerebral embolism, and cerebral hemorrhage.

Further, the neurogenic pulmonary edema is selected from one or more of pulmonary edema caused by craniocerebral trauma, a surgery, subarachnoid hemorrhage, and cerebral embolism.

Further, the neurogenic pulmonary edema is selected from one or more of pulmonary edema caused by craniocerebral trauma, a surgery, and subarachnoid hemorrhage.

Further, the neurogenic pulmonary edema is selected from one or more of pulmonary edema caused by craniocerebral trauma and a surgery.

Further, the neurogenic pulmonary edema is selected from pulmonary edema caused by craniocerebral trauma.

The pharmacological experiments verify that the herba schizonepetae and radix saposhnikoviae granule prepared from the traditional Chinese medicinal composition of the present invention can obviously reduce a lung index and lung wet weight/dry weight of a rat with neurogenic pulmonary edema induced by oleic acid, relieve a pathological state of a lung tissue of the rat, and reduce the degree of lung tissue edema. The pharmacological experiment verify that can further obviously reduce lung wet weight/dry weight of a mouse with neurogenic pulmonary edema induced by spinal cord injury, relieve a pathological state of a lung tissue of the mouse, and reduce the degree of lung tissue edema. The traditional Chinese medicinal composition increases selection of a drug for clinically treating neurogenic pulmonary edema.

### BRIEF DESCRIPTION OF THE DRAWINGS

FIG. 1 shows images of lung tissues of rats in each group of a model of oleic acid-induced acute pulmonary edema; and

FIG. 2 shows images of lung tissues of rats in each group of a model of spinal cord injury-induced neurogenic pulmonary edema.

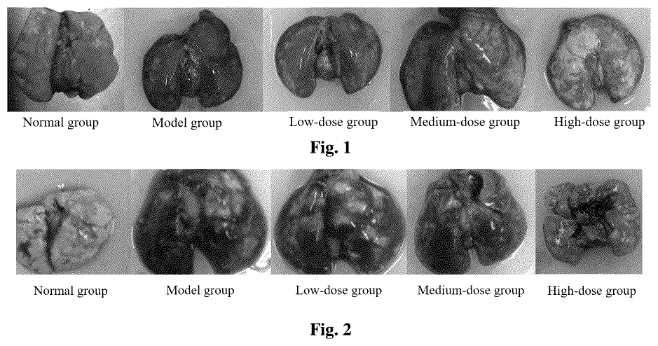

### DETAILED DESCRIPTION OF EMBODIMENTS

### Example 1 Preparation of Granule

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 25% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste with the relative density of 1.30 (80-85° C.), 1 part of the clear paste was taken, 6 parts of sucrose powder were added, the materials were uniformly mixed, the mixture was prepared into a granule, the granule was dried, the volatile oil obtained in step A was added, and the materials were uniformly mixed to obtain a granule.

### Example 2 Preparation of Granule

### Prescription:

50 g of herba schizonepetae, 100 g of radix saposhnikoviae, 50 g of Notopterygium root, 100 g of angelicae pubescentis radix, 15 g of radix bupleuri, 100 g of common hogfennel root, 50 g of rhizoma chuanxiong, 100 g of fructus aurantii, 50 g of poria cocos, 100 g of platycodonis radix, and 5 g of licorice root.

The preparation method was the same as that in example 1.

### Example 3 Preparation of Granule

### Prescription:

100 g of herba schizonepetae, 50 g of radix saposhnikoviae, 100 g of Notopterygium root, 50 g of angelicae pubescentis radix, 100 g of radix bupleuri, 15 g of common hogfennel root, 100 g of rhizoma chuanxiong, 125 g of fructus aurantii, 125 g of poria cocos, 15 g of platycodonis radix, and 40 g of licorice root.

The preparation method was the same as that in example 1.

### Preparation Example 4 Preparation of Oral Liquid

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 10% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, a proper amount of sucrose was added, the materials were uniformly mixed, the volatile oil obtained in step A was added, the materials were uniformly mixed, and water was added to 1,000 ml to obtain an oral liquid.

### Example 5 Preparation of Syrup

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 40% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, a proper amount of sucrose was added, the materials were uniformly mixed, the volatile oil obtained in step A and 500 ml of simple syrup were added, the materials were uniformly mixed, the mixture was stood and filtered, and water was added to 1,000 ml to obtain a syrup.

### Example 6 Preparation of Tablet

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 15% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, a proper amount of sucrose was added, the materials were uniformly mixed, the mixture was prepared into a granule, the granule was dried, the volatile oil obtained in step A was added, the materials were uniformly mixed, the mixture was prepared into a granule, a proper amount of an excipient was added, the materials were uniformly mixed, and the mixture was tableted to obtain a tablet.

### Example 7 Preparation of Capsule

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.
step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 30% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, a proper amount of sucrose was added, the materials were uniformly mixed, the mixture was prepared into a granule, the granule was dried, the volatile oil obtained in step A was added, the materials were uniformly mixed, the mixture was prepared into a granule, the granule was dried and crushed, and the crushed granule was encapsulated to obtain a capsule.

### Example 8 Preparation of Pill

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 25% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, a proper amount of sucrose was added, the materials were uniformly mixed, the mixture was prepared into a granule, the granule was dried, the volatile oil obtained in step A was added, the materials were uniformly mixed, the mixture was dried, crushed and sieved, 40-60 g of refined honey and a proper amount of a water were added to prepare a pill, the pill was dried to obtain a finished product.

### Example 9 Preparation Example of Extract

### Prescription:

75 g of herba schizonepetae, 75 g of radix saposhnikoviae, 75 g of Notopterygium root, 75 g of angelicae pubescentis radix, 75 g of radix bupleuri, 75 g of common hogfennel root, 75 g of rhizoma chuanxiong, 75 g of fructus aurantii, 75 g of poria cocos, 75 g of platycodonis radix, and 25 g of licorice root.

### Preparation Method

step A: herba schizonepetae, radix saposhnikoviae, Notopterygium root, angelicae pubescentis radix, common hogfennel root, rhizoma chuanxiong, and fructus aurantii were respectively distilled to extract a volatile oil for a standby application, and distilled medicinal residues, and distilled rhizoma chuanxiong and fructus aurantii water solutions were collected for a standby application;
step B: the distilled rhizoma chuanxiong and fructus aurantii water solutions obtained in step A were prepared into a 25% ethanol solution for a standby application;
step C: poria cocos, and the distilled rhizoma chuanxiong and fructus aurantii medicinal residues obtained in step A were mixed, the mixture was percolated with the ethanol solution obtained in step B, and a percolate was collected for a standby application;
step D: radix bupleuri, platycodonis radix, licorice root, the distilled herba schizonepetae, radix saposhnikoviae, Notopterygium root, angelicae pubescentis radix, and common hogfennel root medicinal residues obtained in step A were decocted in water twice with 1.5 hours each time, two decoctions were mixed, the mixture was filtered, and a filtrate was concentrated into a thick paste for a standby application; and
step E: the percolate obtained in step C and the thick paste obtained in step D were mixed, the mixture was stood and filtered, a filtrate was concentrated into a clear paste, the volatile oil obtained in step A was added, and the materials were uniformly mixed to obtain an extract.

In order to verify the efficacy of the traditional Chinese medicinal composition of the present invention for treating neurogenic pulmonary edema, the inventor carries out related pharmacodynamic tests. It should be noted that drugs selected in the pharmacodynamic tests described below are obtained by the representative formula of the present invention and a preparation method therefor. The inventor also carries out pharmacodynamic experiments on drugs obtained by other formulas contained in the present invention and preparation methods therefor. The experimental results show that the drugs obtained by other formulas and preparation methods thereof have the same or similar effects, but the drugs are not exhaustive due to space limitations. In addition, the pharmacodynamic experiments described below verify the efficacy of the present invention only by taking a partially representative animal model as an example.

The inventor explains that the following experimental researches are carried out on the basis of an acute toxicity test and a long-term toxicity test for proving the drug safety, and the administration dose in the experimental researches is within a safe dose range.

Oleic acid is a fatty acid with relatively strong toxicity, can stimulate vasoconstriction to cause rising of a pulmonary artery pressure through an intravenous injection, damages vascular endothelial cells, increases the permeability of an alveolus-capillary membrane, causes pulmonary interstitial and alveolar edema, reduces lung compliance and functional residual volume, and forms an alveolar transparent membrane. The use of oleic acid is a mature common molding method of pulmonary edema.

It is a typical molding method of an animal with neurogenic pulmonary edema by performing laminectomy on a rate to cause spine injury and thus inducing pulmonary edema of the mouse. The molding method is simple and easy to implement and high in success rate.

### Pharmacodynamic Example 1 Effects of Herba Schizonepetae and Radix Saposhnikoviae Granule on Rat Model of Oleic Acid-Induced Acute Pulmonary Edema

### 1 Materials

### 1.1 Animals

6-8 week old male Wista rats weighed 200-220 g were provided by the Lunan Pharmaceutical Co., Ltd and adaptively fed for one week before an experiment.

### 1.2 Drugs and Reagents

### 1.2.1 Drugs

Granule of example 1 of the present invention; and

oleic acid.

### 1.2.2 Dose in Rats

Granule of example 1:4 g/kg (low dose), 8 g/kg (medium dose), and 16 g/kg (high dose).

### 2 Experimental Process

### 2.1 Animal Grouping, Molding and Administration

60 rats were taken and randomly divided into a blank group, a model group, an example 1 low-dose group, an example 1 medium-dose group, and an example 1 high-dose group, with 12 rats in each group. The rats in the blank group and the model group were intragastrically administrated with 10 ml/kg of purified water, and the rats in the example 1 low-dose, medium-dose and high-dose groups were respectively intragastrically administrated with 4 g/kg, 8 g/kg and 16 g/kg of a herba schizonepetae and radix saposhnikoviae granule with the administration volume of 10 ml/kg for once a day for 2 consecutive days. After 30 min of the administration on the 3rd day, the tail vein of each group of rats except the blank group was injected with 0.2 ml/kg of oleic acid, timing was immediately carried out, after 6 h, the rats were anesthetized and sacrificed, the lungs were taken out for observation, the lung tissues were weighed, and a lung index and lung wet weight/dry weight were calculated. Lung index=lung weight/body weight; and the lower left lung was weighed as the lung wet weight, and the lower left lung was dried at 70° C. for 48 h to a constant weight as the lung dry weight.

### 2.2 Statistical Processing

Statistical processing was performed using an SPSS 17.0 software, measurement data were expressed as (x ±s [symbol] , a one-way analysis of variance was used for comparison among one group, and a t-test was used for comparison between groups. P<0.05 indicated that the difference was statistically significant.

### 3 Results and Conclusions

The general morphology of the lungs of each group was observed. The lung tissues of the rats in the blank group were normal, smooth in surface, free of extravasated blood, and light pink; the lung tissues of the rats in the model group were obviously enlarged, bloody spots of extravasated blood can be seen, and the lungs were dark red, and when the trachea was cut off, pink foams overflew; and compared with the model group, the rats in the example 1 low, medium and high-dose groups had obviously reduced pulmonary extravasated blood, and a few foams or no obvious foams were shown in the trachea. The experimental results showed that the herba schizonepetae and radix saposhnikoviae granule can effectively relieve pulmonary edema and histopathological changes and had an exact therapeutic effect on the pulmonary edema.

Compared with the blank group, the lung index and the lung wet weight/dry weight were significantly increased in the model group (both P<0.05); and compared with the model group, the lung index was significantly reduced in the example 1 low, medium and high-dose groups (P<0.05 and P<0.01), and the lung wet weight/dry weight was significantly reduced in the example 1 medium and high-dose groups (both P<0.05). The experimental results were shown in Table 1 and indicated that the herba schizonepetae and radix saposhnikoviae granule can significantly relieve pulmonary edema symptoms of the rats.

TABLE 1
Results of herba schizonepetae and radix saposhnikoviae granule
on lung index and lung wet weight/dry weight (x̅ ± s, n = 12)

| | Lung index | Wet/dry |
|---|---|---|
| Groups | (mg/g) | (g/g) |
| Blank group | 6.42 ± 0.72 | 5.54 ± 0.53 |
| Model group | 10.55 ± 1.08** | 6.08 ± 0.33* |
| Example 1 low-dose group | 8.92 ± 1.49^{##} | 5.74 ± 0.65 |
| Example 1 medium-dose group | 9.49 ± 1.46^{#} | 5.62 ± 0.50^{#} |
| Example 1 high-dose group | 8.55 ± 1.31^{##} | 5.60 ± 0.29^{#} |

Note:
compared with the normal group,
*indicated P < 0.05 and
**indicated P < 0.01; and compared with the model group,
^{#}indicated P < 0.05 and
^{##}indicated P < 0.01.

### Pharmacodynamic Example 2 Effects of Herba Schizonepetae and Radix Saposhnikoviae Granule on Rat Model of Oleic Acid-Induced Neurogenic Pulmonary Edema

### 1 Materials

### 1.1 Animals

6-8 week old male SD rats weighed 200-220 g were provided by the Lunan Pharmaceutical Co., Ltd and adaptively fed for one week before an experiment.

### 1.2 Drugs

Granule of example 1 of the present invention; and

oleic acid.

### 1.2.2 Dose in Rats

Granule of example 1:4 g/kg (low dose), 8 g/kg (medium dose), and 16 g/kg (high dose).

### 2 Experimental Process

### 2.1 Animal Grouping, Molding and Administration

50 rats were taken and randomly divided into a blank group, a model group, an example 1 low-dose group, an example 1 medium-dose group, and an example 1 high-dose group, with 10 rats in each group. The rats in the normal group and the model group were intragastrically administrated with of purified water, and the rats in the example 1 low-dose, medium-dose and high-dose groups were respectively intragastrically administrated with 4 g/kg, 8 g/kg and 16 g/kg of a granule of example 1 with the administration volume of 10 ml/kg for once a day for 2 consecutive days. After 30 min of the administration on the 3rd day, the animals were anesthetized and fixed, the parietal skin was incised, a No. 7 syringe needle was vertically inserted into the center of the parietal bone for a depth of about 6 mm, after bloodless withdrawn, 0.2 ml/kg of oleic acid was slowly injected, timing was started, after 10 min, the rats were sacrificed, the lungs were taken out for observation, the lung tissues were weighed, and a lung index and lung wet weight/dry weight were calculated. Lung index=lung weight/body weight; and the inferior lobe of the left lung was weighed as the lung wet weight, the inferior lobe of the left lung was dried at 70° C. for 48 h to a constant weight as the lung dry weight, and the lung wet/dry weight was calculated.

### 2.2 Statistical Processing

Statistical processing was performed using an SPSS 17.0 software, measurement data were expressed as (\hat{x}? s??, a one-way analysis of variance was used for comparison among one group, and a t-test was used for comparison between groups. P<0.05 indicated that the difference was statistically significant.

### 3 Results and Conclusions

The lung tissues of the rats in each group were observed. It was found that the two lungs of the rats in the blank group were full, smooth in surface, and pink; the lungs of the rats in the model group were obviously enlarged, the surfaces of the lungs were dark red, and after the trachea was cut off, a large amount of foams overflew; and compared with the model group, the lesion degrees of the example 1 low, medium and high-dose groups were obviously reduced. The experimental results showed that the herba schizonepetae and radix saposhnikoviae granule can effectively relieve pulmonary edema and histopathological changes and had an exact therapeutic effect on the pulmonary edema.

Compared with the blank group, the lung index and lung wet weight/dry weight were significantly increased in the model group (both P<0.01), which indicated that the molding was successful; and compared with the model group, the lung index and lung wet weight/dry weight were significantly reduced the example 1 medium and high-dose groups (P<0.05 and P<0.01). The experimental results were shown in Table 2 and the experimental results indicated that the herba schizonepetae and radix saposhnikoviae granule can significantly relieve neurogenic pulmonary edema of the rats.

TABLE 2
Effects of herba schizonepetae and radix saposhnikoviae
granule on lung index and lung wet weight/dry weight of
mice with oleic acid-induced neurogenic pulmonary
edema (x̅ ± s, n = 10 )

| | Lung index | Lung wet weight/ |
|---|---|---|
| Groups | (mg/g) | dry weight (g/g) |
| Blank group | 6.32 ± 0.53 | 5.24 ± 0.53 |
| Model group | 9.98 ± 0.98** | 6.10 ± 0.29* |
| Example 1 low-dose group | 9.58 ± 1.25 | 5.71 ± 0.59 |
| Example 1 medium-dose group | 8.63 ± 1.38^{#} | 5.63 ± 0.38^{#} |
| Example 1 high-dose group | 8.48 ± 1.20^{##} | 5.60 ± 0.27^{#} |

Note:
compared with the normal group,
*indicated P < 0.05 and
**indicated P < 0.01; and compared with the model group,
^{#}indicated P < 0.05 and
^{##}indicated P < 0.01.

### Pharmacodynamic Example 2 Effects of Herba Schizonepetae and Radix Saposhnikoviae Granule on Rat Model of Spinal Cord Injury-Induced Neurogenic Pulmonary Edema

### 1 Materials

### 1.1 Animals

4-6 week old male ICR mice weighed 23-25 g were provided by the Lunan Pharmaceutical Co., Ltd and adaptively fed for one week before an experiment.

### 1.2 Drugs

Granule of example 1 of the present invention; and

dexamethasone.

### 2. Experimental Process

### 2.1 Animal Grouping, Molding and Administration

40 mice were taken and randomly divided into a sham surgery group, a model group, a dexamethasone group, an example 1 granule group, and a dexamethasone+example 1 granule group, with 8 mice in each group. The rats in the sham surgery group were subjected to laminectomy without spinal cord injury, while the rats in the model group and each administration group were subjected to laminectomy and spinal cord injury. After molding for 24 h, the mice in the sham surgery group and the model group were intramuscularly injected with normal saline (10 ml/kg) and intragastrically administered with purified water (10 ml/kg) at the same time, the mice in the example 1 granule group were intragastrically administered with an example 1 granule (18 g/kg), the mice in the dexamethasone group were intramuscularly injected with dexamethasone (0.025 mg/kg), and the mice in the dexamethasone+example 1 granule group were intragastrically administered with the example 1 granule (18 g/kg) and intramuscularly injected with dexamethasone (0.025 mg/kg) at the same time. The animals were administered for 2 consecutive days. The animals were anesthetized and sacrificed on the 3rd of the molding, the lungs were taken out, the wet weight was weighed, the lungs were placed in a 60° C. oven for drying for 48 h and taken out, the dry weight was weighed, and a ratio of wet weight/dry weight was calculated.

### 2.2 Statistical Processing

Statistical processing was performed using an SPSS 17.0 software, measurement data were expressed as (\hat{x}? s??, a one-way analysis of variance was used for comparison among one group, and a t-test was used for comparison between groups. P<0.05 indicated that the difference was statistically significant.

### 3 Results and Discussions

Compared with the sham surgery group, the lung wet weight/dry weight was significantly increased (P<0.001) in the model group, which indicated that the molding was successful; and compared with the model group, the lung wet weight/dry weight was significantly reduced in the example 1 granule group and the dexamethasone+example 1 granule group (both P<0.001), and the reduction effect was better than that of the dexamethasone group (P<0.01). The results were shown in Table 3. The experimental results showed that the herba schizonepetae and radix saposhnikoviae granule had a significant protective effect on neurogenic pulmonary edema induced by spinal cord injury, and the drug effect significantly better than that of the dexamethasone.

TABLE 3
Effects of herba schizonepetae and radix saposhnikoviae granule
on lung wet weight/dry weight of mice with spinal cord injury-induced
neurogenic pulmonary edema (x̅ ± s, n = 8 )

| | Lung wet weight/ |
|---|---|
| Grouping | dry weight |
| Sham surgery group | 4.09 ± 0.30 |
| Model group | 5.24 ± 0.35*** |
| Dexamethasone group | 4.92 ± 0.13^{#} |
| Example 1 granule group | 4.57 ± 0.21^{###&&} |
| Dexamethasone + example 1 granule group | 4.33 ± 0.16^{###&&&} |

Note:
Compared with the sham surgery group,
***indicated P < 0.001; compared with the model group,
^{#}indicated P < 0.05,
^{##}indicated P < 0.01, and
^{###}indicated P < 0.001; and compared with the dexamethasone group, ^{&&}indicated P < 0.01 and
^{&&&}indicated P < 0.001.

1. Use of a traditional Chinese medicinal composition consisting of herba schizonepetae, radix saposhnikoviae, Notopterygium root, angelicae pubescentis radix, radix bupleuri, common hogfennel root, rhizoma chuanxiong, fructus aurantii, poria cocos, platycodonis radix, and licorice root in the preparation of a drug for preventing or treating neurogenic pulmonary edema.

2. The use according to claim 1, wherein the neurogenic pulmonary edema is pulmonary edema caused by craniocerebral injury or a central nervous system disease.

3. The use according to claim 1, wherein the neurogenic pulmonary edema is pulmonary edema that occurs after injury to a central nervous system.

4. The use according to claim 1, wherein the neurogenic pulmonary edema is selected from one or more of pulmonary edema caused by craniocerebral trauma, a surgery, subarachnoid hemorrhage, cerebral embolism, cerebral hemorrhage, brainstem infarction, an intracranial tumor, acute cerebral edema, epilepsy, and virus infection combined with a central nervous system lesion.

5. The use according to any one of claims 1-4, wherein the traditional Chinese medicinal composition is mainly prepared from the following raw materials in parts by weight:
5-30 parts of herba schizonepetae, 5-30 parts of radix saposhnikoviae, 5-30 parts of Notopterygium root,
5-30 parts of angelicae pubescentis radix, 3-25 parts of radix bupleuri, 3-25 parts of common hogfennel root,
5-30 parts of rhizoma chuanxiong, 3-25 parts of fructus aurantii, 5-30 parts of poria cocos,
3-25 parts of platycodonis radix, and 1-10 parts of licorice root.

6. The use according to claim 5, wherein the traditional Chinese medicinal composition is mainly prepared from the following raw materials in parts by weight:
10-20 parts of herba schizonepetae, 10-20 parts of radix saposhnikoviae, 10-20 parts of Notopterygium root,
10-20 parts of angelicae pubescentis radix, 5-20 parts of radix bupleuri, 5-20 parts of common hogfennel root,
10-20 parts of rhizoma chuanxiong, 5-20 parts of fructus aurantii, 10-20 parts of poria cocos,
5-20 parts of platycodonis radix, and 3-10 parts of licorice root.

7. The use according to claim 6, wherein the traditional Chinese medicinal composition is mainly prepared from the following raw materials in parts by weight:
15 parts of herba schizonepetae, 15 parts of radix saposhnikoviae, 15 parts of Notopterygium root,
15 parts of angelicae pubescentis radix, 15 parts of radix bupleuri, 15 parts of common hogfennel root,
15 parts of rhizoma chuanxiong, 15 parts of fructus aurantii, 15 parts of poria cocos,
15 parts of platycodonis radix, and 5 parts of licorice root.

8. The use according to any one of claims 1-7, wherein the traditional Chinese medicinal composition can be prepared into one or more of a tablet, a capsule, a pill, a granule, a mixture, and an oral liquid by adding one or more pharmaceutically acceptable pharmaceutic adjuvants.

9. The use according to claim 8, wherein the oral formulation is a granule or a mixture.
